(19) United States
(12) Patent Application Publication
(10) Pub. No.: US 2014/0117051 A1
(43) Pub. Date: May 1, 2014

(54) FLUID PRODUCT DISPENSING HEAD

(71) Applicant: Frederic Duquet, Crespieres (FR)

(72) Inventor: Frederic Duquet, Crespieres (FR)

(73) Assignee: APTAR FRANCE SAS, Le Neubourg (FR)

(21) Appl. No.: 14/123,261

(22) PCT Filed: Jun. 7, 2012
(86) PCT No.: PCT/FR2012/051275

§ 371 Date: Dec. 2, 2013

(30) Foreign Application Priority Data
Jul. 25, 2011 (FR) 1156777

(51) Int. Cl. B67D 1/08

(52) U.S. Cl. CPC B05B 15/02; A61L 2/10
USPC 222/148

(57) ABSTRACT

A fluid dispenser head for associating with a dispenser member, such as a pump, and with a reservoir, so that together they form a fluid dispenser, the head including a fluid dispenser orifice via which the fluid leaves the head so as to be accessible to a user, the head also includes a removable protective cap that masks the dispenser orifice in its storage condition, the dispenser head being characterized in that the cap emits radiation that is suitable for irradiating any fluid residue at the dispenser orifice.

The present invention relates to a fluid dispenser head for associating with a dispenser member, such as a pump, and with a reservoir, so that together they form a fluid dispenser. The head includes a fluid dispenser orifice where a user may recover the dispensed fluid. The head also includes a removable protective cap that masks the dispenser orifice in the storage condition, it being necessary to remove the cap in order to recover the fluid dispensed through the dispenser orifice. The invention also relates to a dispenser comprising a reservoir, a dispenser member, and a dispenser head of the invention. Advantageous fields of application of the present invention are the fields of cosmetics, perfumery, and pharmacy.

Dispenser heads of this type are very frequently used in the field of cosmetics for dispensing viscous fluids such as creams, gels, etc. The fluid leaves the dispenser orifice in the form of a bead or a glob. The user may recover the fluid by means of a finger, or onto another desired application surface. In the present invention, the dispenser orifice is any kind of end opening from which the fluid is accessible by the user. In general, the dispenser orifice is formed in a pusher that the user can move axially down by means of one or more fingers. The dispenser orifice often extends laterally or radially relative to the movement axis of the pusher. Thus, when the pusher is depressed, the dispenser orifice moves axially over a distance that corresponds to the stroke of the pusher. In order to prevent any unintentional or accidental actuation of the pusher and in order to protect the dispenser orifice, the pusher is conventionally covered or masked by a protective cap that is generally in the form of an upsidedown cup. The cap comprises a top wall that is arranged above the pusher, and a side wall that is often cylindrical and that surrounds the pusher. The cap may be held in place on the dispenser at its bottom annular edge that may be snap-fastened on a stationary element of the dispenser, such as the reservoir or a fastener ring. As a result, the dispenser orifice faces the side wall of the cap. The dispenser is then in its storage condition, given that it is not possible to access the pusher masked by the cap. When the user wishes to use the dispenser, it is necessary to begin by removing the cap axially so as to unmask the pusher and make it accessible. The user may then press on the pusher so as to move it axially, thereby causing fluid to be dispensed through the dispenser orifice. Naturally, the user seeks to recover all of the fluid dispensed at the dispenser orifice. However, a residue of fluid always remains in the dispenser orifice and/or around the dispenser orifice. Given that it is extremely difficult to recover the residue of fluid, the user leaves the dispenser in this state and puts the cap back into place. As a result, the residue of fluid dries out and deteriorates, causing micro-organisms, germs, bacteria, microbes, etc. to appear. On the next dispensing operation, the user once again removes the cap and presses on the pusher so as to dispense the fluid through the dispenser orifice. The new dose of fluid naturally comes into contact with the dried-out and deteriorated residue of fluid resulting from the previous dispensing operation. As a result, the new dose of fluid also becomes contaminated by the micro-organisms, germs, bacteria, microbes, etc. resulting from the fluid residue. It is thus not guaranteed that the dispensed fluid is hygienic.

An object of the present invention is to remedy the above-mentioned drawback of the prior art by defining a dispenser head and a dispenser having successive dispensed doses that are not contaminated by residues of fluid resulting from previous dispensing operations. Another object of the present invention is to conserve largely unchanged the general structure of the dispenser head constituted by the dispenser orifice and the cap. Still another object of the invention is to sterilize or to neutralize any residues of fluid without requiring any handling, other than the conventional handling of removing the cap from the dispenser orifice and of putting it back into place.

To achieve the various objects, the present invention proposes a fluid dispenser head for associating with a dispenser member, such as a pump, and with a reservoir, so that together they form a fluid dispenser, the head including a fluid dispenser orifice via which the fluid leaves the head so as to be accessible to a user, the head also includes a removable protective cap that masks the dispenser orifice in its storage condition, the dispenser head being characterized in that the cap emits radiation that is suitable for irradiating any fluid residue at the dispenser orifice. In other words, the cap is used as support for supporting radiation that is directed towards the dispenser orifice that might be contaminated by a residue of fluid. Advantageously, the cap includes a radiation source that emits the radiation towards the dispenser orifice. In a variant, the radiation source may also be positioned remotely in some other component element of the dispenser.

According to another advantageous characteristic of the invention, the radiation source is arranged in the direct proximity of the dispenser orifice. Preferably, the radiation source is arranged immediately facing the dispenser orifice. Thus, all of the power emitted by the radiation is focused on the dispenser orifice. The radiation can reach the fluid residues situated around the dispenser orifice, and also the fluid inside the dispenser orifice.

In a practical embodiment, the cap is movable in an axial direction, the dispenser orifice being oriented transversally to the axial direction. This is a conventional configuration in which the cap is removed in the same axial direction as the downwards and upwards movement of the pusher. Advantageously, the cap is oriented angularly relative to the dispenser orifice so as to constrain the radiation to be positioned to face the dispenser orifice. In this way, it is guaranteed that the radiation is always arranged facing the dispenser orifice, since the user does not have any other choice in positioning the cap.

In another practical embodiment, the dispenser orifice is formed by a pusher that is movable downwards and upwards in an axial direction. Once again, this is a conventional configuration in which the dispenser orifice is constrained to move with the pusher. The dispenser orifice is generally arranged in transverse or radial manner, but it is also possible to envisage arranging the dispenser orifice in the axial direction of movement of the pusher.

In another advantageous aspect, the cap incorporates trigger means for triggering the radiation, which trigger means are sensitive to the cap being put into place over the dispenser orifice. Advantageously, the trigger means comprise a presence detector inside the cap. The presence detector may function with or without contact. In addition, the cap may incorporate timer means that are suitable for interrupting the radiation at the end of a determined period of time. Thus, when the cap is put into place around the dispenser orifice, the trigger means trigger the emission of radiation towards the dispenser orifice for a period of time that is determined by the timer means. Thus, the user does not even need to worry about activating and de-activating the radiation that serves to irradiate any fluid residue situated at the dispenser orifice. Conventional handling remains unchanged.

In an advantageous embodiment, the radiation is an ultra-violet radiation, advantageously emitted by a light-emitting diode (LED), having a wavelength in the range about 253 nanometers (nm) to 254 nm, so as to decontaminate or sterilize any fluid residue at the dispenser orifice. By using another type of radiation, it is possible to act on the fluid residues or even on the dose of dispensed fluid for some other purpose.

The invention also provides a fluid dispenser comprising: a fluid reservoir; a dispenser member, such as a pump, mounted on the reservoir; and a dispenser head of the invention, mounted on the dispenser member; the orifice being formed by a pusher that is movable downwards and upwards in an axial direction, the orifice being oriented transversally to the axial direction of movement of the pusher, the cap being mounted on a stationary portion of the dispenser, masking the pusher. Advantageously, the pusher is prevented from turning, the cap being indexed in turning so as to constrain the radiation to be positioned to face the dispenser orifice, while the cap is being put into place on the pusher.

One of the principles of the present invention is to use the protective cap that covers the dispenser orifice as a support or transmission member for supporting or transmitting radiation that sterilizes or de-contaminates any fluid residues situated at the dispenser orifice. Indexing the cap relative to the dispenser orifice makes it possible to constrain the radiation to take up correct positioning relative to the dispenser orifice.

The invention is described more fully below with reference to the accompanying drawing, which shows an embodiment of the invention by way of non-limiting example.

In the figures:

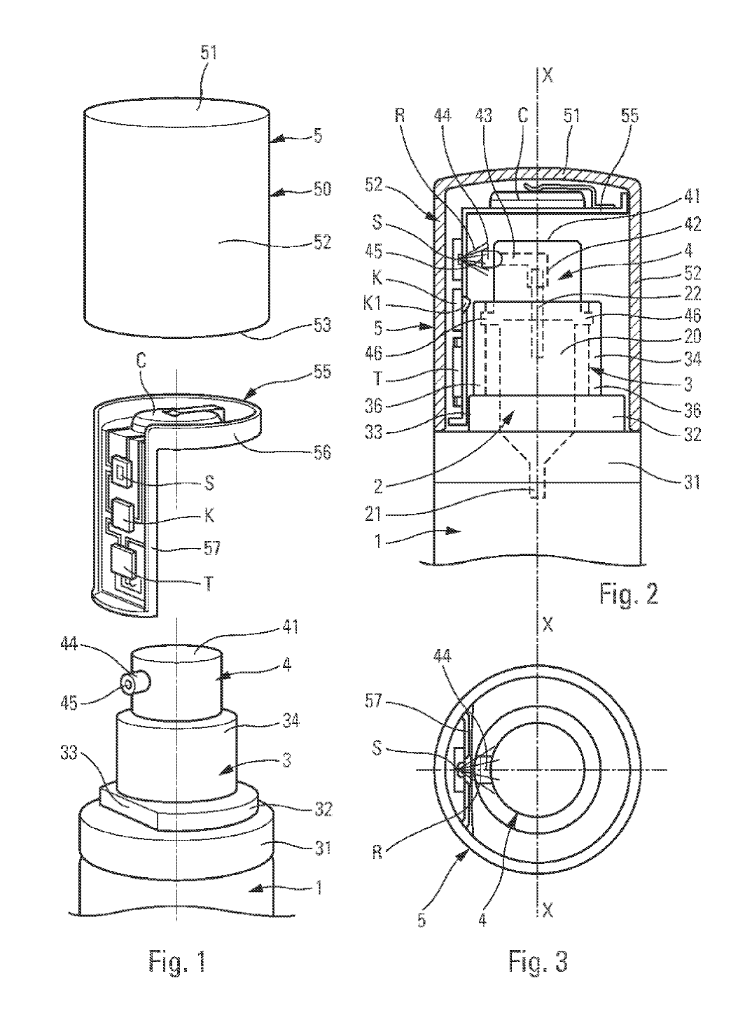

FIG. 1 is an exploded perspective view of a dispenser in a non-limiting embodiment of the invention;

FIG. 2 is a vertical section view through the FIG. 1 dispenser; and

FIG. 3 is a horizontal cross-section view through the FIGS. 1 and 2 dispenser at the dispenser orifice.

The fluid dispenser shown in the figures is more particularly for viscous fluids, such as creams, gels, etc. It essentially comprises five component elements, namely: a reservoir 1; a dispenser member 2, which is a pump; a fastener ring 3 for fastening the pump on the reservoir; a pusher 4 that is mounted on the pump; and a cap 5 that incorporates the present invention; except for the cap 5, and to a lesser extent the fastener ring 3, the other component elements, namely the reservoir 1, the pump 2, and the pusher 4 may be of design that is entirely conventional.

The fluid reservoir 1 may be of any kind, of any shape, and made of any appropriate material. It defines an internal fluid storage volume that may be constant or variable. In the field of cosmetics, variable-capacity reservoirs are generally used so that the fluid stored therein does not come into contact with the outside air. Naturally, the reservoir includes an opening that puts its internal volume into communication with the outside.

As mentioned above, the dispenser member 2 is a manual pump that includes a pump body 20 defining an inlet 21 that is in communication with the reservoir 1. The pump 2 also includes an actuator rod 22 that is axially movable down and up inside the body 20, in such a manner as to cause the volume of a pump chamber to vary so as to put a dose of fluid under pressure. In FIG. 2, it can be seen that the actuator rod 22 extends along the axial direction X, which also constitutes an axis of symmetry, or indeed an axis of revolution, for the dispenser. Although not shown, the actuator rod 22 is provided with a piston for sliding in leaktight manner inside a slide cylinder of the pump chamber. The operation of the pump is entirely conventional: by driving the actuator rod 22 into the pump body 20, the fluid contained in the pump chamber is put under pressure in such a manner as to be forced up through the actuator rod. When the actuator rod 22 is released, it returns to its rest position under the action of a return spring, and fluid from the reservoir is sucked into the pump chamber through the inlet 21.

The main function of the fastener ring 3 is to hold the pump 2 relative to the reservoir 1. Fastening is preferably permanent and leaktight. The fastener ring 3 comprises a bottom section 31 that engages the opening of the reservoir 1, an intermediate section 32, and a top section 34. In FIG. 1, it should be observed that the intermediate section 32 includes a flat 33 that interrupts its circular shape. As described below, the flat 33 serves to index the cap 5 relative to the remainder of the dispenser. The pump 2 is held, by any appropriate means, in stationary and leaktight manner inside the fastener ring 3. In FIG. 2, it should be observed that the pump body extends through the three sections of the fastener ring. The actuator rod 22 may project axially upwards out from the fastener ring 3.

The pusher 4 is mounted on the free end of the actuator rod 22, and may be moved downwards and upwards in the axial direction X. In this way, the pusher 4 drives the actuator rod 22 into the pump body 20. The pusher 4 includes a connection sleeve 42 that is engaged around the free end of the actuator rod 22, the sleeve communicating with an endpiece 44 via an internal delivery duct 43. The endpiece 44 forms a dispenser orifice 45 that can be seen in FIG. 1. It should be observed that the endpiece 44 projects radially or laterally outwards, such that the dispenser orifice 45 is oriented transversally, and more particularly perpendicularly, relative to the axial direction X. Thus, when the pusher 4 is moved axially downwards and upwards, the dispenser orifice 45 is also constrained to move with the pusher 4. While moving, the pusher 4 penetrates, in part, into the top section 34 of the fastener ring 3.

The protective cap 5 includes a cap body 50 that is preferably opaque. The cap body 50 presents a general configuration in the shape of an upsidedown cup, thus defining a top wall 51 and a substantially-cylindrical side wall 52 that defines an annular bottom edge 53. Once in place on the dispenser, as shown in FIG. 2, the top wall 51 is arranged above the pusher 4, and the side wall 52 extends around the pusher 4 and the fastener ring 3. More precisely, the side wall 52 surrounds the intermediate section 32 and the top section 34 of the fastener ring: the bottom annular edge 53 of the cap 5 coming to bear on the bottom section 31 of the fastener ring 3. Snap-fastening may be provided between the cap 5 and the fastener ring 3, so as to hold the cap 5 securely on the dispenser in its storage condition.

The above-described dispenser presents a design that is entirely conventional in the fields of cosmetics, perfumery, and pharmacy. The pusher 4, and more particularly its dispenser orifice 45, associated with the cap 5 constitute a fluid dispenser head in the broadest sense. Without going beyond the ambit of the invention, it is possible to disassociate the dispenser orifice from the pusher 4, e.g. so as to make the orifice stationary relative to the reservoir 1. The dispenser orifice 45 may thus be connected to the pusher 4 via a flexible hose. Other dispenser configurations also make it possible to disassociate the dispenser orifice from the pusher 4. In the context of the present invention, the dispenser head should be understood as the association of a dispenser orifice with a protective cap. In the particular non-limiting embodiment shown in the figures, the dispenser head is constituted by the pusher 4 and the cap 5.

In the invention, the protective cap 5 includes a support element 55 that is arranged inside the space formed by the cap. By way of example, the support element 55 may form a top plate 56 and a side tab 57 that are connected together at an edge of the plate, such that the tab 57 extends axially downwards. Thus, the support element 55 may be inserted inside the cap body 50 so that the plate 56 comes to be positioned immediately below the top wall 51, and the tab 57 immediately against the side wall 52. This is clearly visible in FIG. 2 and also understandable from FIG. 1. The bottom end of the tab 57 extends down substantially as far as the bottom end of the side wall 52 of the cap 5, so as to make it possible to position it against the flat 33 of the intermediate section 32 of the fastener ring 3. Thus, the support element 55 constrains the protective cap 5 to take up a particular angular orientation relative to the fastener ring 3, and as a result relative to the remainder of the dispenser. The cap 5 is thus always oriented in the same way relative to the dispenser. In addition, the pusher 4 is prevented from turning relative to the remainder of the dispenser, such that the endpiece 44 and its dispenser orifice 45 are always oriented in the same way. By way of example, in order to block the pusher 4, it is possible to provide two guide lugs 46 on the pusher 4 that slide in two axial grooves 36 that are formed in the top section 34 of the fastener ring 3. This is a conventional characteristic that is frequently used to prevent the pusher from turning. Thus, the cap 5 is indexed relative to the dispenser and the dispenser orifice 45 is prevented from turning, thereby constraining the dispenser orifice 45 to occupy a particular position relative to the cap 5. As can be seen in FIGS. 2 and 3, the dispenser endpiece 44 is oriented towards the tab 57 that has a bottom end that comes into contact with the flat 33. This constitutes the only possible position for the cap 5 relative to the dispenser and the dispenser orifice.

In the invention, the support element 55 supports a radiation source S that is suitable for emitting radiation R for irradiating the dispenser orifice 45 and its close surroundings. In this way, if any residual fluid remains at the dispenser orifice and/or around the dispenser orifice, it is irradiated by the radiation R. It can be seen in FIGS. 2 and 3 that the source S and its radiation R are arranged in the proximity of, and immediately facing, the dispenser orifice 45. The source S is arranged on a printed circuit card that is mounted on the tab 57. In order to activate the source S, trigger means K are provided, e.g. in the form of a switch including a trigger member K1 that may be mechanical and/or electronic. By way of example, provision may be made for the trigger member K1 to come into contact with the top section 34 of the fastener ring 3. The trigger member K1 may also be provided in the form of a presence detector that detects the presence of an object inside the cap. The trigger member K1 may thus function with or without direct contact. The trigger means K are advantageously associated with timer means T that are suitable for interrupting the radiation R at the end of a determined period of time. By way of example, the timer means may act on the trigger means K so as to reinitialize them. By way of example, it is possible to provide radiation duration of about 10 seconds to about 1 minute. For electrically powering the system, it is possible to provide a battery C that may be arranged at the plate 56. Once the support element 55 is equipped in this way, it is inserted into the cap body 50, with all of the electronic elements arranged between the support element 55 and the cap body so that no electronic element can be seen.

The protective cap 5 is handled in exactly the same way as a conventional protective cap. It is put into place and removed by moving it axially in the direction X. When it is put into place, the trigger member K1 detects the presence of the pusher 4 and/or of the fastener ring 3, or, in a variant, it comes into direct contact with the pusher 4 and/or the fastener ring 3, so as to trigger radiation R from the source S. During this operation, the user does not intervene in any way in order to trigger and operate the source S. The radiation R is thus emitted for a determined period of time by the timer means T. At the end of this period of time, the radiation R is stopped. The protective cap 5 then once again provides no more than a conventional protection function. When the user removes the cap 5 axially, the source S remains inactive: it is only while the cap is being put back into place on the dispenser that the source emits its radiation once again for a determined period of time. Consequently, the cap is handled in entirely conventional manner: The user may even be unaware of the presence of the radiation source and of the associated electronic components.

In an application of the present invention, the radiation R is ultra-violet radiation, having a wavelength in the range about 253 nm to 254 nm, that is suitable for performing a decontamination or a sterilization function. By way of example, the radiation may be emitted by an LED. With an intensity in the range 6 millijoules per square centimeter (mJ/cm²) to 40 mJ/cm², it is guaranteed that 99.99% of most bacteria that could develop in fluid residues in the zone of the dispenser orifice 45 will be destroyed. Naturally, the intensity depends on the power of the source S, on the distance from the source to the orifice, and on the irradiation time of the radiation R. The fluid residues that accumulate at the dispenser orifice and/or around the dispenser endpiece 44 are thus decontaminated and/or sterilized, such that the next-dispensed dose of fluid is not contaminated by the fluid residues resulting from prior dispensing operations.

In the above-described embodiment, the source of radiation S is carried by the cap 5. In a variant that is not shown, it is also possible to envisage positioning the source remotely in another component element of the dispenser, such as the fastener ring 3 for example, and to convey the radiation by means of a waveguide or an optical fiber to the dispenser orifice. The same applies for the associated electronic elements, such as the trigger means K and the timer means T, and the battery C, that could be housed inside the fastener ring 3. A principle of the invention is to use the cap 5 as a support for supporting radiation emitted directly onto the dispenser orifice and its close surroundings.

According to another optional characteristic of the invention, the surface of the head around the dispenser orifice includes a bactericidal photocatalyst, the radiation irradiating the bactericidal photocatalyst. Thus, the function of the radiation is to activate or to stimulate the effect, or the bactericidal properties, of a photosensitive substance that in turn acts on the fluid present on the surfaces of the head so as to sterilize or decontaminate them. The surface may extend to all of the head and even to the cap. The radiation, advantageously emitted by an LED, has a wavelength in the range about 280 nm to 380 nm, so as to activate the bactericidal effect of the photocatalyst. The bactericidal photocatalyst may be titanium dioxide TiO₂. The bactericidal photocatalyst is applied to the surface or is incorporated in a wall forming the surface.

1. A fluid dispenser head for associating with a dispenser member, such as a pump, and with a reservoir, so that together they form a fluid dispenser, the head including a fluid dispenser orifice via which the fluid leaves the head so as to be accessible to a user, the head also includes a removable protective cap that masks the dispenser orifice in its storage condition, the dispenser head being characterized in that the cap emits radiation that is suitable for irradiating any fluid residue at the dispenser orifice.

2. A dispenser head according to claim 1, wherein the cap includes a radiation source that emits the radiation towards the dispenser orifice.

3. A dispenser head according to claim 2, wherein the radiation source is arranged in the direct proximity of the dispenser orifice.

4. A dispenser head according to claim 2, wherein the radiation source is arranged immediately facing the dispenser orifice.

5. A dispenser head according to claim 1, wherein the cap is movable in an axial direction, the dispenser orifice being oriented transversally to the axial direction.

6. A dispenser head according to claim 5, wherein the cap is oriented angularly relative to the dispenser orifice so as to constrain the radiation to be positioned to face the dispenser orifice.

7. A dispenser head according to claim 1, wherein the dispenser orifice is formed by a pusher that is movable downwards and upwards in an axial direction.

8. A dispenser head according to claim 1, wherein the cap incorporates trigger means for triggering the radiation, which trigger means are sensitive to the cap being put into place over the dispenser orifice.

9. A dispenser head according to claim 8, wherein the trigger means comprise a presence detector inside the cap.

10. A dispenser head according to claim 1, wherein the cap incorporates timer means that are suitable for interrupting the radiation at the end of a determined period of time.

11. A dispenser head according to claim 1, wherein the radiation is an ultra-violet radiation, advantageously emitted by a light-emitting diode, having a wavelength in the range about 253 nm to 254 nm, so as to decontaminate or sterilize any fluid residue at the dispenser orifice.

12. A fluid dispenser comprising:
a fluid reservoir;
a dispenser member, such as a pump, mounted on the reservoir; and
a dispenser head according to claim 1, mounted on the dispenser member;
the orifice being formed by a pusher that is movable downwards and upwards in an axial direction, the orifice being oriented transversally to the axial direction of movement of the pusher, the cap being mounted on a stationary portion of the dispenser, masking the pusher.

13. A dispenser according to claim 12, wherein the pusher is prevented from turning, the cap being indexed in turning so as to constrain the radiation to be positioned to face the dispenser orifice, while the cap is being put into place on the pusher.
